(19) United States
(12) Patent Application Publication
(10) Pub. No.: US 2010/0137447 A1
(43) Pub. Date: Jun. 3, 2010

(54) METHOD FOR THE PRODUCTION OF ADSORBATES OF A RASAGILINE SALT HAVING A WATER-SOLUBLE ADJUVANT

(75) Inventors: Alexander Lehmann, Zurchel (DE); Frank Muskulus, Laupheim (DE); Julia Schulze-Nahrup, Neuried (DE)

Correspondence Address: SMITH PATENT CONSULTING, LLC, 3307 DUKE STREET, ALEXANDRIA, VA, 22314, US

(73) Assignee: ratiopharm GmbH, Ulm (DE)

(21) Appl. No.: 12/598,301

(22) PCT Filed: Apr. 30, 2008
(86) PCT No.: PCT/EP2008/003498

§ 371 Date: Nov. 24, 2009

(30) Foreign Application Priority Data
Apr. 30, 2007 (EP) 07008755.6

(51) Int. Cl. A61K 31/135; A61P 25/16; A61P 25/28; A61P 25/18

(52) U.S. Cl. 514/657

(57) ABSTRACT

The present invention relates to an adsorbate of a pharmaceutically compatible rasagiline salt comprising at least one pharmaceutically compatible adjuvant, wherein the at least one pharmaceutically compatible adjuvant is a water-soluble, organic solvent and the rasagiline salt is present in the adsorbate as an amorphous substance.

The present invention relates to novel adsorbates of pharmaceutically compatible salts of the known active ingredient rasagiline with water-soluble, pharmaceutically compatible, organic adjuvants and methods for the production thereof. In these novel adsorbates, the pharmaceutically compatible salt of the rasagiline is present in an amorphous form which is stable and does not convert to a different, for example crystalline, form of the pharmaceutically compatible salt of rasagiline even upon lengthy storage after incorporation of the adsorbate into a pharmaceutical product.

Rasagiline is the designation for the chemical compound R(+)-N-propargyl-1-aminoindane which is also called R(+)PAI.

Rasagiline is an MAO inhibitor known for a long time which is used as an active ingredient in the treatment of a number of diseases, For example, U.S. Pat. No. 5,532,415 discloses the preparetion of rasagiline and its salts as well as the application of the active ingredient in the treatment of a number of diseases, e.g. Parkinson's disease, memory disorders, dementia, depression, schizophrenia, hyperactivity, etc. A method for preparing salts of rasagiline is also disclosed in WO 02/068376.

Even though rasagiline and its salts have been described as pharmaceutical active ingredients for quite some time, the formulation of pharmaceutical products with pharmaceutically compatible salts of rasagiline poses problems. For example, U.S. Pat. No. 6,126,968 describes that pharmaceutical formulations with PAI which may be present either as a racemate or as a (+)- or (−)-enantiomer suffer from stability problems. To solve these problems, U.S. Pat. No. 6,126,968 proposes to formulate the active ingredients with a very large quantity of an organic alcohol such as, especially, mannitol. WO 2006/091657 reports problems resulting from the fact that the homogeneous distribution of a rasagiline salt in a pharmaceutical product required by the law on pharmaceutical products cannot be fully guaranteed. In order to solve this problem, WO 2006/091657 proposes to use active ingredient particles of a certain size.

It is also becoming increasingly important in the field of the formulation of pharmaceutical products that the physical form of an active ingredient (i.e. the polymorphous or amorphous form) does not change during storage of the pharmaceutical formulation and that the pharmaceutical products contain the active ingredient in a defined and reproducible polymorphous or amorphous form. The pharmaceutically compatible rasagiline salts are commonly present in the crystalline form; therefore, the occurrence of polymorphous substances is highly likely. If such polymorphous forms are incorporated into pharmaceutical products, it is advantageous to use a pure polymorphous form, and it must be ensured that no conversion between the individual polymorphous substances in the pharmaceutical product occurs. Therefore, it would be advantageous to provide rasagiline in an amorphous form which may be reproduced easily, is amenable to processing and does not convert to other forms. However, it has not been possible so far to reproducibly provide the pharmaceutically compatible salts of rasagiline in a stable amorphous form which is suitable for formulating pharmaceutical products.

Therefore, it is the object of the invention to provide pharmaceutically compatible salts of rasagiline in such a form that, after incorporation into a pharmaceutical product, the active ingredient will remain stable throughout the storage period of the pharmaceutical product, that no conversions to other physical forms of the active ingredient occur and that homogeneity of the distribution of the active ingredient in the pharmaceutical products can be ensured in a simple manner. In addition, the preparation and processing of the form of the pharmaceutically compatible salts of rasagiline should be easy.

This object is achieved by the surprising finding that, after removal of the solvent, aqueous solutions comprising a pharmaceutically compatible salt of rasagiline and a water-soluble, organic, pharmaceutically compatible adjuvant form adsorbates between the pharmaceutically compatible salt of rasagiline and the pharmaceutically compatible organic adjuvant where the pharmaceutically compatible salt of rasagiline is present in an amorphous form. These adsorbates may be processed easily, are highly stable in pharmaceutical formulations, especially in tablets, capsules, pellets and granulate, and they provide pharmaceutical products with high homogeneity in the distribution of the active ingredient. In particular, the amorphous form of the rasagiline salts does not convert to other physical forms after incorporation into pharmaceutical products and during the storage thereof.

Therefore, the invention provides an adsorbate of a pharmaceutically compatible salt of rasagiline comprising at least one pharmaceutically compatible adjuvant, said at least one pharmaceutically compatible adjuvant being a water-soluble, organic adjuvant and the salt of rasagiline being present as an amorphous substance in the adsorbate. These adsorbates may be obtained by a process wherein a pharmaceutically compatible salt of rasagiline and at least one pharmaceutically compatible, water-soluble, organic adjuvant are dissolved in an aqueous medium and the aqueous medium is then removed. The invention also provides a process for preparing the adsorbates and pharmaceutical products containing these adsorbates. The following observations apply both to the adsorbates of the invention and to the process for the preparation thereof.

The adsorbates of the invention preferably do not contain an inorganic adjuvant.

During preparation, pharmaceutically compatible salts of rasagiline are generally present as a crystalline substance. When these salts are dissolved in water (or an aqueous solvent) and the water is then removed (for example by spray drying), crystalline or partially crystal-line substances are obtained in turn. It has now been surprisingly found that, in the event that a pharmaceutically compatible rasagiline salt is dissolved with an organic, water-soluble, pharmaceutically compatible adjuvant and the water is then removed, for example by spray drying, an adsorbate between the pharmaceutically compatible rasagiline salt and the organic, water-soluble, pharmaceutically compatible adjuvant is obtained wherein the pharmaceutically compatible rasagiline salt is present in an amorphous form.

In this invention, an amorphous form is interpreted to mean a form of the active ingredient wherein the diffraction reflexes of the active ingredient no longer show in an X-ray diffractogram. For details regarding the determination of the X-ray diffractogram, reference is made to the following examples.

The pharmaceutically compatible rasagiline salts are not subject to special limitations; any of the customary pharmaceutically compatible salts may be used for this purpose. Preferred salts are the tartrates, the esylates, the mesylates, the edisilates and the sulfates. The mesylate salt of rasagiline is most preferred.

In a preferred embodiment of the invention, the adsorbate consists of the pharmaceutically compatible rasagiline salt and one or more, preferably one, pharmaceutically compatible, water-soluble, organic adjuvant. In this embodiment, the adsorbate does not contain any water-insoluble components and no inorganic components.

In another preferred embodiment, the adsorbate consists of the pharmaceutically compatible rasagiline salt and one or more, preferably one, pharmaceutically compatible, organic, water-soluble adjuvant and one or more, preferably one, water-insoluble adjuvant. Preferably, the water-insoluble adjuvant is also an organic compound; the presence or inorganic compounds in the adsorbates is not preferred for the invention.

If water-insoluble, pharmaceutically compatible adjuvants are components of the adsorbate, these are preferably water-insoluble, pharmaceutically compatible adjuvants having a particle size of 150 μm or less, preferably 100 μm or less, especially preferably in the range of approx. 50 μm. The lower limit of the particle size of the pharmaceutically compatible, water-insoluble adjuvants is about 10 μm, more preferably about 20 μm. Preferably, this adjuvant is microcrystalline cellulose, especially preferably microcrystalline cellulose having a mean particle size of 150 μm or less, more preferably 100 μm or less, especially about 50 μm. Especially preferably, said microcrystalline cellulose is Avicel PH101.

An essential component of the adsorbates of the invention is at least one pharmaceutically compatible adjuvant which is a water-soluble organic adjuvant. The adsorbate of the invention preferably contains a water-soluble, organic, pharmaceutically compatible adjuvant, but it is also possible to use several water-soluble, pharmaceutically compatible, organic adjuvants, for example two, three or four such adjuvants.

The pharmaceutically compatible, water-soluble, organic adjuvants are preferably water-soluble polymers such as those used as binders for pharmaceutical products in the prior art, e.g. cellulose ether or polyvinyl pyrrolidone. Especially preferred are hydroxypropyl methyl cellulose, hydroxypropyl cellulose or polyvinyl pyrrolidone. If such a water-soluble polymer is used as the water-soluble, organic, pharmaceutically compatible adjuvant, the adsorbate preferably does not comprise any water-insoluble adjuvant.

Another preferred water-soluble adjuvant is an organic acid such as an organic mono-, di- or tricarboxylic acid having up to 10 carbon atoms which, optionally and preferably, contains one to three hydroxyl groups. The most preferred organic, water-soluble acid is citric acid. If the organic, pharmaceutical, water-soluble adjuvant is not a water-soluble polymer, but, for example, an organic, water-soluble acid such as citric acid, it is preferred to use this organic, water-soluble, pharmaceutically compatible adjuvant together with a water-insoluble, pharmaceutically compatible adjuvant as defined above. It goes without saying that any salt of this acid may be used instead of the organic acid.

The ratio between the pharmaceutically compatible rasagiline salt and the at least one pharmaceutically compatible, water-soluble, organic adjuvant in the adsorbate is preferably in the range of 5:1 to 1:20, more preferably in the range of 2:1 to 1:15. It is most preferred that the amount of the pharmaceutically compatible, water-soluble, organic adjuvant is at least as high as or higher than the amount of the pharmaceutically compatible rasagiline salt so that the ratio between the salt and the adjuvant is most preferably in the range of 1:1 to 1:10.

If more than one pharmaceutically compatible, water-soluble, organic adjuvant is present in the adsorbate, the above statements regarding the ratio between the pharmaceutically compatible rasagiline salt and the at least one pharmaceutically compatible, water-soluble organic adjuvant also apply for the preferred ratios between the pharmaceutically compatible rasagiline salt and the total of all the pharmaceutically compatible, water-soluble, organic adjuvants in the adsorbate. If one or more water-insoluble, pharmaceutically compatible adjuvants are present in the adsorbate of the invention, the ratio between the pharmaceutically compatible rasagiline salt and the one or more (preferably one) pharmaceutically compatible, water-insoluble adjuvants is preferably in the range of 5:1 to 1:20, more preferably in the range of 2:1 to 1:15, and most preferably in the range of 1:1 to 1:10.

Unless expressly disclosed otherwise or evident to a person skilled in the art from the circumstances, any ratios, percentages and contents specified in this application are based on the weight.

An adjuvant within the meaning of the present application is water-soluble if more than 0.1 mg of the adjuvant may be dissolved in 1 ml of water. Preferably, a water-soluble adjuvant is soluble to such an extent that more than 1 mg, more preferably more than 10 mg, especially more than 100 mg, e.g. 1000 mg or more may be dissolved in 1 ml of water.

An adjuvant within the meaning of the present application is water-insoluble if not more than 0.1 mg of the active ingredient may be dissolved in 1 ml of water.

In each case, the solubilities are based on distilled water at 25° C. and pH=7.

The adsorbates of the invention may be prepared in a simple manner by dissolving a pharmaceutically compatible rasagiline salt and the at least one organic, water-soluble, pharmaceutically compatible adjuvant as well as, optionally, other water-soluble components of the adsorbate in water or an aqueous solvent. If water-insoluble components should be present in the adsorbate, these water-insoluble components are dispersed in the solvent subsequently (or in advance). Then the solvent is removed, preferably by spray drying. Such spray drying may be conducted in the customary manner, for example with a Büchi spray dryer of the type B-191, for example at a temperature of 130° C. and a spray rate of 5, 10 or 20%. However, the spray drying conditions are not critical as long as no temperature is used at which the rasagiline decomposes during the spraying period.

In the invention, an aqueous solvent is interpreted to mean a solvent at least 50%, more preferably 70%, of which consist of water, the remainder consisting of one or more water-miscible solvents, especially of an alcohol such as ethanol or isopropanol. It is preferred to use water as the solvent in the method of the invention to prepare the adsorbates of the invention.

The adsorbates of the invention may be processed in the customary manner to obtain pharmaceutical products. If desired, the adsorbates may be ground to an advantageous particle size and screened to an advantageous particle distribution.

The adsorbates of the invention may generally be designed for oral, parenteral, rectal or transdermal administration, preferably oral administration. Suitable forms of administration, for example, are tablets (prepared by granulation or direct compression), tablets disintegrating in the mouth, delayed-release tablets, pellets or granulates that may, for example, be introduced into coated tablets, sachets, hard or soft gelatine capsules, etc. Processing to obtain tablets, pellets or granules is carried out as described in the prior art and known to a person skilled in the art. For example, reference may be made to the standard textbook “Die Tablette”, Wolfgang A. Ritschel and Anette Bauer-Brandl, 2^{nd} ed., Editio Cantor Verlag, 2002.

The tablets, pellets or granules of the invention, may, for example, comprise suitable carrier substances (e.g. fillers), binders, lubricants, disintegrants, dyes, flavouring agents, fluidisers, film-coats and, optionally, fluxing agents and other customary adjuvants and additives. Suitable carriers, for example, are pellets on a sugar basis and microcrystalline basis, lactose, alactose monohydrate, gelatine, agar, starch, corn starch, sucrose, glucose, methyl cellulose, dicalcium phosphate, calcium phosphate, calcium silicates, mannitol, sorbitol, microcrystalline cellulose, cellulose derivatives, fatty acid, fats, stearates, oils, waxes, paraffins, etc. Suitable binders comprise starch, gelatine, natural sugars such as glucose or R-lactose, starch, gum Arabic, tragacanth, sodium alginate, povidone, carboxymethyl cellulose, polyethylene glycol, wax, hydroxypropyl methyl cellulose, hydroxypropyl cellulose etc. Examples of lubricants are sodium oleate, sodium stearate, magnesium stearate, sodium benzoate, sodium acetate, sodium chloride, stearic acid, sodium stearyl fumarate, talcum powder, etc. Disintegrants include the well-known “super disintegrants”, but starch, methyl cellulose, agar, bentonite, xanthan gum, pyrrolidones, etc. are also worth mentioning. Especially preferred are sodium crosscarmelose and sodium starch glycolate. Suitable film coats and functional enteric and retarding film coats are celluloses and derivatives thereof, polyglycols, povidones, acryl polymers, copolymers on the basis of ethyl acrylate, methyl methacrylate, polyvinyl acetate and methacrylic acid. In addition, the pharmaceutical products may, for example, comprise suitable dyes on the basis of iron oxide.

It was already known to formulate pharmaceutically compatible rasagiline salts with the above adjuvants. These adjuvants also include pharmaceutically compatible, water-soluble, organic compounds. However, the adsorbates of the invention are not the result of processing these pharmaceutically compatible, water-soluble, organic compounds with a pharmaceutically compatible rasagiline salt and are therefore novel compounds. In order to obtain such adsorbates it is not sufficient to process the pharmaceutically compatible rasagiline salts with adjuvants in the usual manner to obtain pharmaceutical products. Rather, a solution of the pharmaceutically compatible rasagiline salt and the pharmaceutically compatible, water-soluble, organic adjuvants that are to yield the adsorbate must be formed and the water then removed to yield the adsorbate. The adsorbates prepared in this manner are novel and have not been described before in the prior art.

The following examples will illustrate the invention.

In the examples, the degree of amorphism was tested by X-ray diffractometry. These measurements are carried out on a Bruker Advance D8, with a Theta-2 Theta goniometer (435 mm diameter) in reflexion geometry (Bragg-Brentano). The radiation power of the X-ray tube (copper anode Kα1, λ=1,5406 Å/Kα2, λ=1,54439 Å) is selected at a generator output of 40 KV/40 mA, and a divergence aperture of 0.6 mm. As a position sensitive detector PSD, a V\dot{a}ntec-1 Detector (12° 2θ detector aperture angle) is used. X-ray diffractions are taken in the range from 3° 2θ to 55° 2θ, at a step distance of 0.016° 2θ (steps 2465) step time: 75 ns. A secondary nickel filter (0.1 mm) is used to filter the Cu-Kβ radiation (λ=1,39222 Å). Primary and secondary Soller gap (2.5°), anti-scattering aperture 5.59 (fixed, distance to the detector window=110.8 mm). Detector aperture (static) 10.28 mm, distance to the detector window=21.3 mm).

The samples are introduced by a horizontal sample changer (9 samples). In order to verify that the measuring array operates correctly, a Korund standard sample is measured before each series of measurements. The intensities (α1/α2=0.5) and reflex positions as well as the α1-α2 reflex distances of this measurement are superimposed with a reference diffraction diagram. This evaluation and the evaluation of the results of the sample measurements as well as the further processing of the raw data is made with the programme package EVA (Bruker).

Rasagiline mesylate prepared in accordance with U.S. Pat. No. 5,532,415 was used for the following experiments.

### REFERENCE EXAMPLE 1

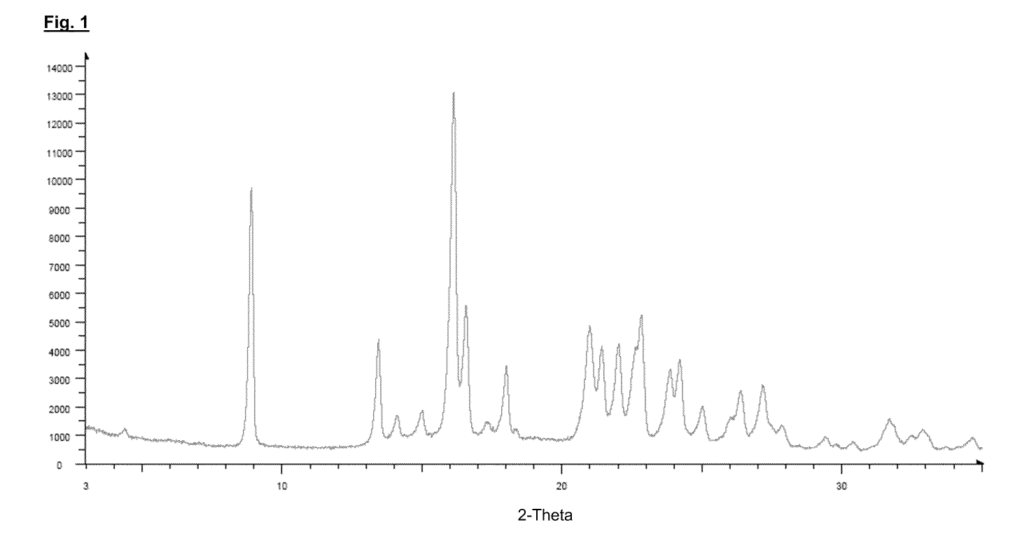

2 g of rasagiline mesylate were dissolved in 50 g of water in a beaker and spray dried with a Buchi type B-191 spray dryer. The following were selected: spray drying temperature 130° C., spray rate 10%, aspirator power 75% and flow control (spray pressure) 50%. The product thus obtained was then examined by X-ray diffraction. FIG. 1 shows the diffractogram.

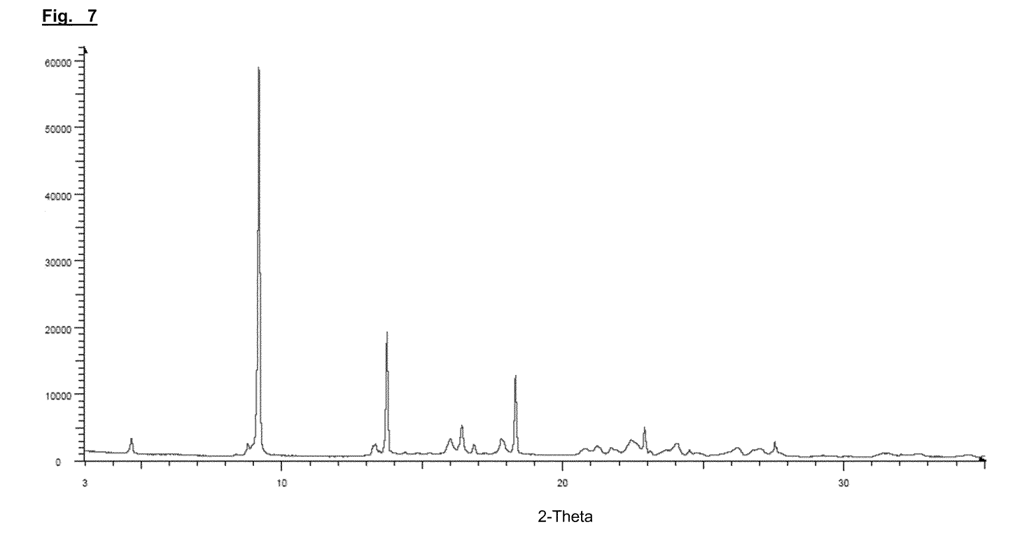

The usual X-ray diffraction peaks of a crystalline rasagiline mesylate are visible. For comparison, a typical rasagiline mesylate X-ray diffractogram is shown in FIG. 7.

### EXAMPLE 1

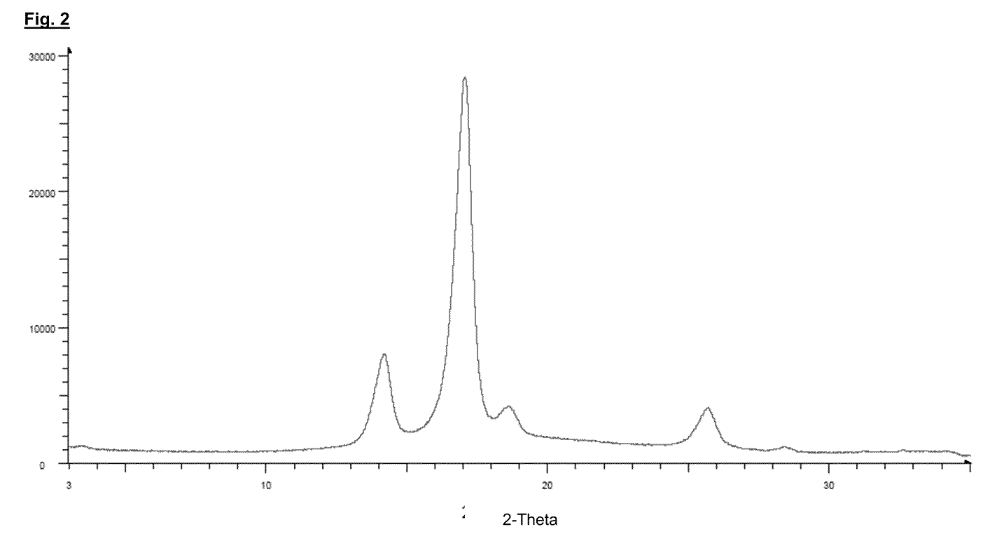

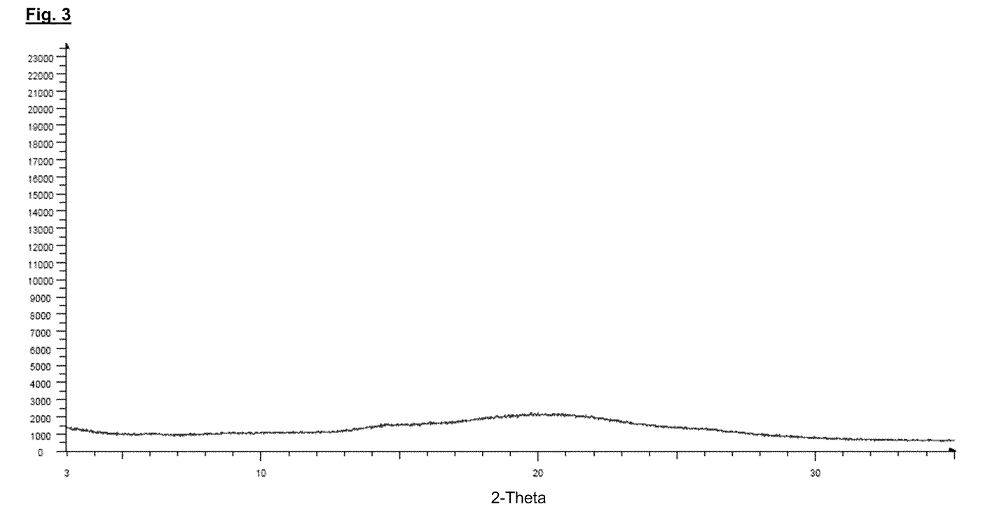

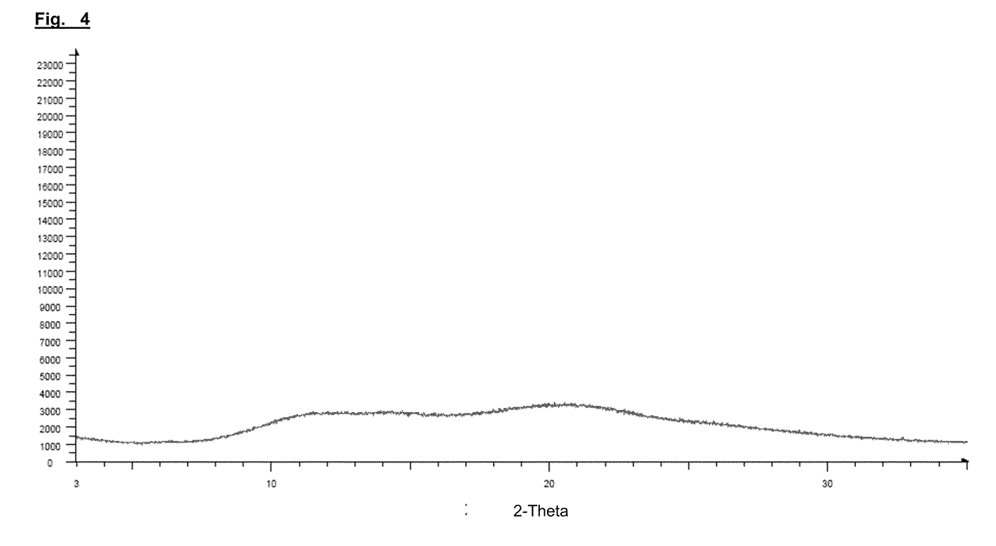

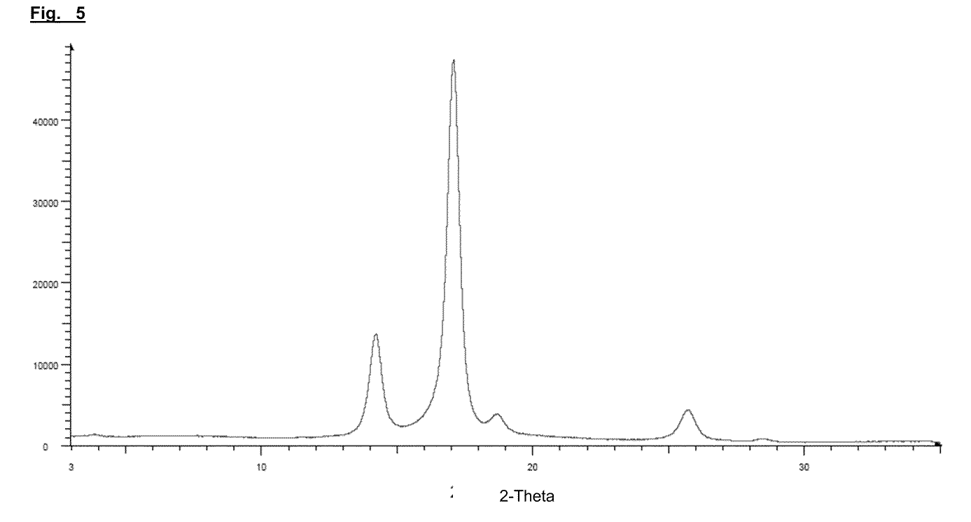

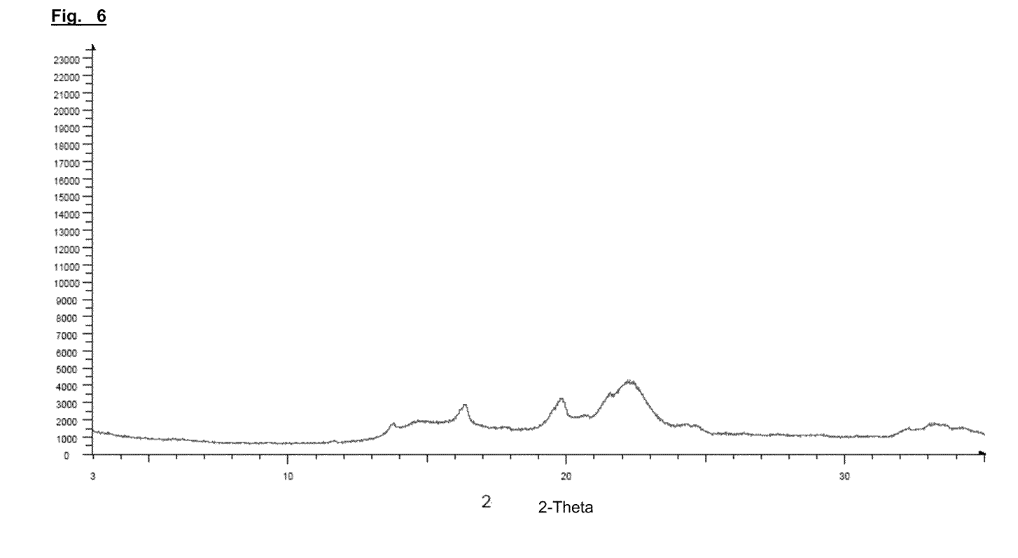

Adsorbates of rasagiline with different adjuvants were prepared in accordance with the following table:

| Example | a | b | c | d | e | f |
|---|---|---|---|---|---|---|
| Rasagiline mesylate | 1 g | 1 g | 1 g | 1 g | 1 g | 1 g |
| Kollidon 25 | 1 g | — | — | 9 g | — | — |
| HPMC | — | 1 g | — | — | 9 g | — |
| Citric acid | — | — | 1 g | — | — | 9 g |
| Avicel PH101 | — | — | 1 g | — | — | 9 g |
| X-ray diffractogram | FIG. 2 | FIG. 3 | — | FIG. 4 | FIG. 5 | FIG. 6 |

In each of the experiments according to example 1a, 1b, 1d, and 1e the amount of rasagiline mesylate indicated in the above table and the indicated amount of water-soluble adjuvant were dissolved in 50 g of water in a beaker and spray dried with the Büchi type B-191 spray drier. The selected spray drying temperature was 130° C., the spray rate 10%, the aspirator power 75%, and the flow control (spray pressure) 50%.

The indicated amount of Avicel PH 101 was used additionally in the experiments 1c and 1f. In that case, the experiment was carried out as follows: The amount of rasagiline mesylate indicated in the above Table and the indicated amount of water-soluble adjuvant are dissolved in 50 g of water in a beaker. Then the water-insoluble adjuvant Avicel PH 101 as described in the above Table was added and mixed in. Following that, the aqueous solutions or mixtures, respectively, were spray dried with the Büchi B-191 spray dryer. The spray drying temperature selected was 130° C., the spray rate 10%, the aspirator power 75% and the flow control (spray pressure) 50%.

Kollidon 25 is a water-soluble polyvinyl pyrrolidone. Hypromellose (HPMC) was selected as the cellulose derivative. The commercial trade name of the citric acid used is “Zitronensaure, wasserfrei, Granulat” (anhydrous, granulate).

X-ray diffraction diagrams as shown in the Figures were taken of the adsorbates.

It was possible to show that the mesylate salt of rasagiline is present in amorphised form in the adsorbates; there is no trace of the typical X-ray diffraction peaks.

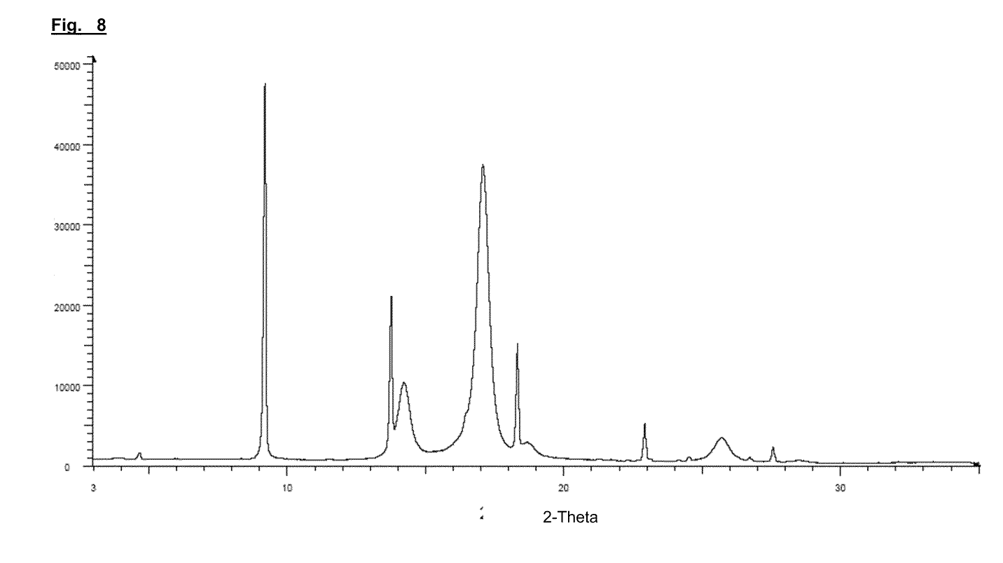

Example 1a (FIG. 2) was fixed to a substrate with glue and tested, because there was not enough amorphous substance. The peaks evident here are caused by the substrate. This was confirmed by the examination of crystalline rasagiline mesylate applied to the same substrate (FIG. 8).

### EXAMPLE 2

Example 2 describes a selected process of preparing a solid dosage form comprising a rasagiline mesylate adsorbate of example 1d.

### Composition:

| No. | Substance | Function | [mg] |
|---|---|---|---|
| 1 | Rasagiline mesylate | API | 3.27 |
| | adsorbate | | |
| 2 | Avicel PH 101 | Filler | 184.80 |
| 3 | Starch 1500 | Disintegrant | 10.00 |
| 4 | Aerosil R972 | Flow control agent | 1.20 |
| 5 | Mg Stearate | Lubricant | 2.00 |
| 6 | Total | | 201.27 |

### Method of Preparation:

Weigh in rasagiline mesylate adsorbate, Avicel PH 101 and Starch 1500 and mix for 5 minutes in a Turbula at 23 rpm. Then screen the mixture through a hand sieve of 0.5 mm and mix for 5 minutes. Add Aerosil R972 and mix in the Turbula for 5 minutes. Screen the mixture through a hand sieve 0.5 mm and mix for 5 minutes. Add magnesium stearate and mix in the Turbula for 5 minutes. Compress to 8 mm cores with EK0.

### EXAMPLE 3

Example 2 was repeated with the difference that the adsorbate of example 1a was used instead of the adsorbate of example 1d. The 3.27 mg of rasagiline adsorbate correspond to 1 mg of rasagiline base.

### EXAMPLE 4

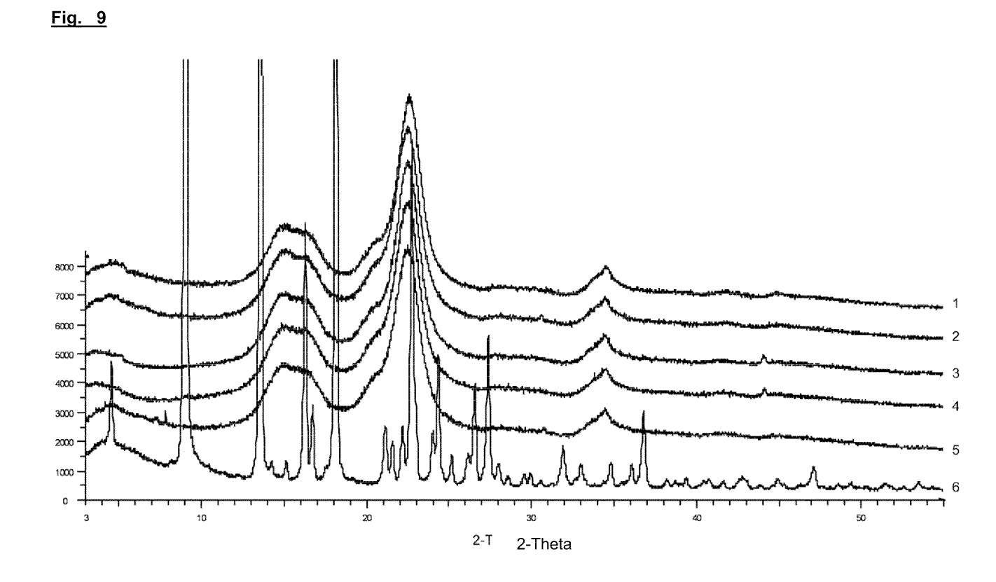

Rasagiline tablets according to example 3 were stored in open or sealed glass bottles, respectively, for up to 12 weeks at 40° C. and 75% relative humidity (RH). At certain points in time, samples were taken and the appearance, hardness, disintegration time and water content (Kart-Fischer titration) of the tablets determined. In addition, the content of the active ingredient and its optical purity as well as the content of related substances was determined by HPLC. In addition, it was investigated by X-ray diffractometry (XRPD) whether the amorphous active ingredient converted to different physical forms. The active ingredient turned out to be sufficiently stable, especially with a view to its physical form. FIG. 9 shows X-ray diffractograms of samples at the beginning of the experiment (5) and after 4 (4) and 12 (2) weeks of storage at 40° C., 75% RH in a sealed container and after 4 (3) and 12 (1) weeks of storage at 40° C., 75% RH in a open container as well as of crystalline rasagiline mesylate (6). It turned out that no crystallisation of the rasagiline salt occurs even after storage.

1. A method for preparing an adsorbate containing a pharmaceutically compatible rasagiline salt present as an amorphous substance and at least one pharmaceutically compatible, water-soluble, organic adjuvant, wherein the adsorbate is prepared by dissolving the pharmaceutically compatible rasagiline salt and at least one pharmaceutically compatible, water-soluble organic adjuvant in an aqueous medium and then removing the aqueous medium.

2. The method according to claim 1, wherein the aqueous medium is removed by spray drying the solution of the pharmaceutically compatible rasagiline salt and the at least one pharmaceutically compatible, water-soluble, organic adjuvant.

3. A method according to claim 1, wherein the pharmaceutically compatible rasagiline salt is the mesylate salt.

4. The method according to claim 1, wherein the adsorbate consists of the pharmaceutically compatible rasagiline salt and one or more pharmaceutically compatible, water-soluble, organic adjuvants.

5. The method according to claim 1, wherein the adsorbate consists of the pharmaceutically compatible rasagiline salt, one or more pharmaceutically compatible, water-soluble, organic adjuvants and one or more pharmaceutically compatible, water-insoluble adjuvants.

6. The method according to claim 5, wherein said pharmaceutically compatible, water-insoluble adjuvant is microcrystalline cellulose.

7. The method according to claim 1, wherein the at least one water-soluble, organic, pharmaceutically compatible adjuvant is selected from the group consisting of water-soluble, pharmaceutically compatible cellulose ethers, water-soluble polyvinyl pyrrolidone and water-soluble, pharmaceutically compatible organic acids.

8. The method according to claim 7, wherein the at least one water-soluble, organic, pharmaceutically compatible adjuvant is selected from the group consisting of hydroxypropyl methyl cellulose, hydroxypropyl cellulose, polyvinyl pyrrolidone and citric acid.

9. The method according to claim 1, wherein the ratio between the pharmaceutically compatible rasagiline salt and the at least one pharmaceutically compatible, water-soluble, organic adjuvant ranges from 5:1 to 1:20.

10. The method according to claim 9, wherein the ratio between the pharmaceutically compatible rasagiline salt and the at least one pharmaceutically compatible water-soluble, organic adjuvant ranges from 1:1 to 1:10.

11. An adsorbate containing a pharmaceutically compatible rasagiline salt present as an amorphous substance and at least one pharmaceutically compatible, water-soluble, organic adjuvant, obtained by the method of claim 1.

12. A pharmaceutical product comprising the adsorbate according to claim 11 and, optionally, one or more pharmaceutically compatible adjuvants and additives.

13. The product according to claim 12, wherein said product takes the form of a capsule, tablet, a tablet disintegrating in the mouth, a delayed-release tablet, pellets or a granulate.
